(19) United States
(12) Patent Application Publication
(10) Pub. No.: US 2009/0286246 A1
(43) Pub. Date: Nov. 19, 2009

(54) Methods for Identifying Compounds that Affect Expression of Cancer-Related Protein Isoforms

(75) Inventors: John Hood, San Diego, CA (US); Charlene F. Barroga, San Diego, CA (US); Dennis Carson, La Jolla, CA (US); Desheng Lu, San Diego, CA (US)

Correspondence Address: FISH & RICHARDSON P.C., PO BOX 1022, MINNEAPOLIS, MN, 55440-1022, US

(73) Assignee: WINTHERIX LLC, San Diego, CA (US)

(21) Appl. No.: 12/437,327

(22) Filed: May 7, 2009

Related U.S. Application Data
(60) Provisional application No. 61/051,324, filed on May 7, 2008.

(51) Int. Cl. C12Q 1/68; C12Q 1/66; C07K 16/18

(52) U.S. Cl. 435/6; 435/8; 530/387.7

(57) ABSTRACT

Provided herein are methods for screening compounds for their ability to modulate the expression of certain isoforms of proteins that are associated with cancer, such as isoforms of proteins that participate in Wnt signaling in cancer cells.

### CROSS-REFERENCE

This application claims the benefit of U.S. Provisional Application No. 61/051,324, filed May 7, 2008, which application is incorporated herein by reference.

The invention relates to assays for screening compounds for their affects on the expression of particular protein isoforms.

### BACKGROUND OF THE INVENTION

The Wnt signaling pathway, which affects cell proliferation and differentiation, is active in certain tissues during embryonic development in mammals, and is also active in many cancers, including colon cancer, leukemias, breast cancer, hepatocellular carcinoma, prostate cancer, and melanoma.

In the canonical Wnt pathway, binding of Wnt, a secreted glycoprotein, to the Frizzled receptor leads to accumulation of beta-catenin in the cytoplasm, resulting in its translocation to the nucleus where it binds to the HMG binding proteins of the LEF/TDF family to activate transcription of Wnt target genes. In the absence of Wnt signaling, beta-catenin is continuously degraded by the ubiquitin pathway; the turnover of beta-catenin is mediated by the beta-catenin destruction complex, which includes the proteins adenomatous polyposis coli (APC), GSK3-beta, and axin. GSK3-beta phosphorylates beta-catenin, marking it for degradation. During Wnt signaling, the beta-catenin destruction complex is disrupted, such that beta catenin phosphorylation is prevented, so that beta-catenin accumulates and then enters the nucleus, where it binds to members of the LEF/TCF family of HMG DNA binding proteins.

While the LEF/TCF family members LEF-1, TCF-1, and TCF-4 do not themselves activate transcription, they do have the ability to bind and bend DNA via their HMG domains. In at least some cases, LEF/TCF proteins bind DNA and recruit transcriptional repressors in the absence of beta-catenin. During Wnt signaling, when beta-catenin becomes available in the nucleus, the repressors are displaced by beta-catenin, which mediates interactions with transcriptional activators. Gene targets of the Wnt pathway include c-myc, cyclin D1, cdx, MMP7, c-myb, c-kit, PPARsigma, axin2, sp5, Bcl-X, LEF-1 itself, and others.

LEF-1, TCF-1, and TCF-4 are alternatively spliced genes. Splice variants of these DNA binding proteins lead to variants having different domains in their C-terminal tails (J. Cell Sci 120: 385-393 (2007)). In addition, both LEF-1 and TCF-1 have dual promoters: each has a first promoter that directs expression of a transcript encoding a full length protein and a second promoter within a downstream intron of the gene that directs expression of an N-terminally truncated version. The N-terminally truncated versions of LEF-1 and TCF-1 (deltaN-LEF-1 and deltaN-TCF-1) lack the beta-catenin binding domain of these proteins but retain their DNA binding domains, allowing these isoforms of LEF-1 and TCF-1 to act as dominant negatives and downregulate the canonical Wnt signaling pathway.

### SUMMARY OF THE INVENTION

Provided herein are methods for screening compounds for their ability to modulate the expression of certain isoforms of proteins that are associated with cancer, such as isoforms of proteins that participate in Wnt signaling in cancer cells.

In one aspect, a method is provided for identifying a compound that modulates a cancer-associated alternative splicing process, in which the method includes: providing a cell that comprises a nucleic acid construct, in which the nucleic acid construct includes a transcription unit that has a promoter and an alternative splice module in which the alternative splice module includes at least three exons, in which the sequences of the exon-intron boundaries of the alternative splice module are derived from a gene that affects or is affected by a signaling pathway that is deregulated in cancer. The transcription unit also includes two differently detectable reporter genes. The alternative splice construct is configured such that when the alternative splice construct is transcribed, two alternative splicing events can occur. A first alternative splicing event results in the splicing of the first exon to the second exon, and splicing of the second exon to the third exon, resulting in the expression of the first reporter gene. A second alternative splicing event results in the splicing of the first exon to the third exon, resulting in the expression of the second reporter gene but not the first reporter gene. The method includes contacting the cell having the alternative splicing construct with a test compound, detecting the a signal from expression of the first reporter gene and a signal from expression of the second reporter gene, and calculating a ratio of the expression of the first reporter gene to the second reporter gene. The difference between the first and second reporter gene expression ratio in the cell contacted with the test compound to the first and second reporter gene expression ratio in a cell not contacted with the test compound are compared, and a difference in the reporter gene expression ratio of test compound-contacted cells with respect to control cells identifies the test compound as a compound that modulates a cancer-associated alternative splicing process.

In some embodiments of the method, the first reporter gene is embedded in-frame within exon 2 of the alternative splicing construct, and the second reporter gene is embedded in-frame within exon 3 of the alternative splicing construct. In these embodiments, a splicing event that joins exons 1, 2, and 3 results in expression of a two reporter gene protein, in which both reporter genes give a detectable signal. The reporter genes can be any reporter genes that have distinguishable signals, for example, two fluorescent protein with different emissions wavelengths, two luciferases with different emissions wavelengths, a luciferase and a fluorescent protein (with distinguishable emissions wavelengths), a luciferase and beta-galactosidase, a luciferase and beta-lactamase, a luciferase and an alkaline phosphatase, etc.

In some embodiments of the method, the first reporter gene and the second reporter gene are both inserted in tandem into exon 3, or at the 3′ end of exon 3. In these embodiments, a first splicing event results in expression of a first reporter protein (and not the second reporter protein), and a second splicing event results in expression of a second reporter protein (and not the first reporter protein) due to a difference in reading frame of the two reporter proteins. The first splicing event that joins exons 1, 2, and 3 results in expression of a protein in which the first reporter gene is out-of-frame (but lacking stop codons), and the second reporter gene is in-frame, producing a detectable signal. The second splicing event that joins exons 1 and 3 results in expression of a protein in which the first reporter gene is in-frame, producing a detectable signal, and the second reporter gene is out-of-frame, producing no signal.

The splicing assay constructs can include more than three exons, for example, the splicing assay constructs can include 4, 5, 6, or more exons, in which the intron/exon boundaries of the exons are derived from a gene that encodes a protein that participates in Wnt signaling. In some embodiments of the methods, a splicing assay construct includes an alternative splice module that includes 4, 5, 6, or more exons, in which the intron/exon boundaries of the exons are derived from a gene that encodes a protein that participates in Wnt signaling, and a reporter gene is embedded in each of the exons of the alternative splice module. In some embodiments, at least two of the reporter genes of the splice module are differently detectable. In preferred embodiments, all of the reporter genes of the splice module are differently detectable.

In some embodiments, the exon-intron boundaries of the alternative splice module are derived from a Bcl-X gene or a Ron gene. In some preferred embodiments, the exon-intron boundaries of the alternative splice module are derived from a gene that affects or is a target of Wnt signaling, such as, for example, Disheveled, LEF-1, TCF-4, or TCF-1.

The method can be performed using cancerous cells, such as cancer cells in which the Wnt signaling pathway is activated, or in noncancerous cells. In some embodiments, noncancerous cells used in the methods include an additional construct that includes a gene encoding a Wnt activator or Wnt modulator. The introduced Wnt activator or modulator gene is operably linked to an inducible or constitutive promoter. In some embodiments, a cell used in the assay methods of the invention is contacted with a Wnt protein.

In another aspect, a method is provided for identifying a compound that increases the expression of the dominant negative form of LEF-1, in which the method includes: providing a cancerous cell that comprises a reporter gene regulated by the P2 promoter of the LEF1 gene, contacting the cell with a test compound, and detecting an increase in the signal from expression of the reporter gene in the cell contacted with the test compound as compared with the expression of the reporter gene in a control cell not contacted with the test compound to identify a compound that promotes transcription of the dominant negative form of LEF1.

In yet another aspect of the invention, a method for identifying a compound that affects the expression of an isoform of a protein that participates in Wnt signaling is provided, in which the method includes: providing a cell that comprises a dual promoter reporter gene construct that includes a promoter region of a gene that produces transcriptional isoforms of gene, in which the promoter region includes two alternative promoters, in which a different isoform of the gene is transcribed from each of the two alternative promoters. The dual promoter construct includes two differently detectable reporter genes operably linked to the dual promoter region of the gene that affects Wnt signaling, and is configured such that expression of the first reporter gene is the result of transcription from the first alternative promoter and expression of the second reporter gene is the result transcription from the second alternative promoter of the dual promoter region. The cell having the dual promoter reporter gene construct is contacted with a test compound, and the signal from expression of the first reporter gene and the second reporter gene is detected. The method further includes identifying a test compound that changes the ratio of expression of the first reporter gene to expression of the second reporter gene with respect to the ratio of expression of the first and second reporter genes in cells that are not contacted with the test compound, to identify a compound that affects expression of a transcriptional isoform of a gene.

In some preferred embodiments, at least one of the transcriptional isoforms of the gene is related to cancer. In some preferred embodiments, the gene encodes a protein that participates in Wnt signaling. In some embodiments, the gene is LEF1, TCF1, or Bcl-X.

### BRIEF DESCRIPTION OF THE DRAWINGS

FIG. 1 shows a construct for a screening assay to detect splicing efficiency of LEF1 exon 11.

FIG. 2 shows a screening assay for splicing efficiency of TCF₄ exon IX.

FIG. 3 show promoter assay to detect expression from P₁ and P₂ promoters of LEF₁.

### INCORPORATION BY REFERENCE

All publications, patents, and patent applications mentioned in this specification are herein incorporated by reference for the subject matter for which they are cited.

### DETAILED DESCRIPTION OF THE INVENTION

### Definitions

As used herein, a cancerous cell or cancer cell is a leukemia cell or a cell derived from a cancerous tumor. One test for whether a nonleukemia cell is cancerous is whether an inoculum of the cells in a nude mouse causes a tumor or tumors. As used herein, a “normal” cell is a noncancerous cell. A normal or noncancerous cell is not derived from a cancerous tumor or leukemia.

“Wnt signaling” or “Wnt pathway signaling” refers to a cell signaling pathway that results in the expression of genes regulated by the interaction of beta catenin with a TCF/LEF protein, such as TCF-1, TCF-3, TCF-4, or LEF-1.

A “protein that participates in Wnt signaling” or a “Wnt-related protein” can be a Wnt activator, a Wnt modulator, or a Wnt target gene. A “Wnt-related gene” is a gene that encodes a protein that participates in Wnt signaling. Proteins that participate in Wnt signaling include, without limitation, Wnt activators (proteins that promote or inhibit beta catenin-TCF/LEF interaction that leads to Wnt target gene expression), including Wnt, Frizzled, Disheveled, LRP5/LRP6 (BMC Genomics 7: 148 (2006)), axin-1 (BMC Genomics 7: 148 (2006)), beta-catenin (BMC Genomics 7: 148 (2006)), axin-2, adenomatous polyposis coli (APC), GSK3-beta (BMC Genomics 7: 148 (2006)); and Wnt modulators (proteins that modulate Wnt target gene expression), including TCF-1 (J. Biol. Chem. 267: 8530-8536 (1992); Mol. Cell. Biol. 16: 745-752 (1996), TCF-3, TCF-4 (J. Biol. Chem. 278: 16169-16175 (2003)); LEF-1 (Nucl. Acids Res. 28: 1994-2003 (2000); Devel. Dynamics 232: 969-978 (2005), CtBP1, Grouch, Pygo, PITX2, and others.

Wnt target genes include, without limitation LEF-1, c-myc, cyclin D1, cdx, MMP7, c-myb, c-kit, PPARsigma, axin2, Bcl-X, sp5, siamois, and others.

“TCF/LEF” refers to any one of TCF-1, TCF-3, TCF-4, or LEF-1, or any combination of two or more of TCF-1, TCF-3, TCF-4, or LEF-1.

As used herein a “Wnt-responsive promoter” is a promoter that is regulated by the interaction of a TCF/LEF protein and β-catenin. The promoter may be regulated by other factors in addition to a TCF/LEF protein and β-catenin. Examples of Wnt-responsive promoters include, but are not limited to, the promoters of the following genes: LEF-1, TCF1, c-myc, c-kit, MMP7, axin2, sp5, cyclinD1, cdx, Bcl-X, and siamois.

As used herein, RNA “isoforms” or transcript isoforms or isoform transcripts are RNA molecules generated by alternative splicing of the same gene. The sequences of the transcript therefore differ. Protein isoforms are translated from RNA isoforms, and have different primary sequences.

“Nucleic acid molecule construct”, “Nucleic acid construct”, “gene construct”, “reporter gene construct”, “splicing construct”, “transcription construct”, “construct”, “recombinant DNA molecule” all refer to nucleic acid molecules that have been isolated and manipulated to excise, join, delete, mutate, expand, extend, replicate, or recombine certain nucleic acid sequences that may be isolated from organisms, replicated from nucleic acid templates isolated from organisms, synthesized, or derived from organisms and synthetic nucleic acid fragments. In the methods of the invention, cells that comprise, include, carry, or have nucleic acid molecules or nucleic acid constructs are cells that have been transformed, transfected, or infected (e.g., with a virus) such that they contain a previously isolated nucleic acid molecule or recombinant nucleic acid molecule or gene construct.

The methods provided herein are used to identify compounds that modulate Wnt signaling in cancer cells. The methods use cell-based assays in which the activity of reporter genes regulated by Wnt signaling-responsive promoters in response to test compounds are compared to the effects of test compounds on noncancerous cells, or to the effects of the test compounds on cells in which the Wnt signaling pathway is not activated by the introduction of induction of Wnt activators or Wnt modulators in the cells.

### Assay Formats

The cell-based assays provided herein can be performed in any feasible format, but are preferably high throughput assays for screening large numbers of compounds. Preferably, the assays are performed in multiwell dishes, such as, for example dishes with 96, 384, or more wells, where each well holds from about 5×10³ to 10⁵ cells, typically from about 10⁴ to 5×10⁴ cells. In preferred embodiments, the assays are performed using reporter genes, in which the signal from the reporter gene is detected by, for example, a luminometer or fluorometer that reads multiwell plates. Plate readers that include an automated dispensing device (for example, for adding reagent buffer for signal detection) are also preferred.

For assays in which cells are transiently transfected with reporter gene constructs or Wnt activator or modulator gene constructs, addition of test compound is typically added 24-48 hours after transfection. In assays in which expression of a gene is induced, for example, by addition of an inducer such as tetracycline or doxycycline, test compound can be added before, at the same time as, or after the inducer. For example, a test compound can be added from 0 to 30 minutes, from 30 minutes to one hour, from one to two hours, from two to three hours, from three to four hours, from four to six hours, from six to eight hours, from eight to ten hours, from 10 to 12 hours, from 12 to 16 hours, from 16 to 20 hours, from 20 to 24 hours, or from 24 to 48 hours after the addition of an inducer.

Reading of the reporter gene signal(s) can be at any time point after the addition of compound, for example, 30 minutes, between 30 minutes and one hour, between one and two hours, between two and three hours, between three and four hours, between four and six hours, between six and eight hours, between eight and ten hours, between 10 and 12 hours, between 12 and 16 hours, between 16 and 20 hours, between 20 and 24 hours, or between 24 and 48 hours after the addition of compound.

Test compounds may be used at a concentration of from about 10 picomolar to about 10 micromolar, for example, from about 1 nanomolar to about 1 micromolar. Initial screens may be performed at a concentration of, for example 100 nanomolar to 10 micromolar, and subsequent secondary screens can be performed at a higher or lower concentration, or at a range of concentrations.

Cellular assays can also be performed to determine the effect of test compounds on the metabolic state, proliferation, growth, or viability of the cells. One or more of a viability assay, cell division assay, cell cycle assay, migration assay, invasion assay, cell death assay, or apoptosis assay, can be performed on the cells in addition to the reporter gene readout assays described herein. For example, cell growth can be monitored using an MTT assay (e.g., the VYBRANT® MTT cell proliferation assay kit, Invitrogen Corp., Carlsbad, Calif.) or BrdU incorporation (the ABSOLUTE-S™ SBIP assay (Invitrogen Corp.). Cell viability (or cytotoxicity) can be assayed by measuring intracellular ATP levels (the ATPLITE™-M kit (Perkin Elmer) or glucose-6-phosphate activity (the Vibrant cytotoxicity assay (Invitrogen Corp.) or by assays using a membrane permeable dye (DiOc 18). In some embodiments, cellular assays are performed in a separate secondary screen. In some embodiments, cellular assays are performed simultaneously with reporter gene assays. For example, assays for viability that use Alamar blue (Nasiry et al., Human Reprod 22: 1304-1309 (2007)) or assays for apoptosis that detect caspase activity (e.g., the APOALERT® caspase assay kits available from Clontech, Mountain View, Calif.), can be performed in the same wells in which reporter gene expression is assayed, provided that the cellular assay readout is distinguishable from the reporter gene expression readout.

### Cells

A cancerous cell used in the methods can be any cancerous cell, and can be, as nonlimiting examples, a colon cancer cell, a leukemia cell, a lymphoma cell, a melanoma cell, a breast cancer cell, a prostate cancer cell, a hepatocarcinoma cell, a lung cancer cell, an ovarian cancer cell, a uterine cancer cell, a cervical cancer cell, or a head-and-neck cancer cell. Nonlimiting examples of leukemia cells include Jurkat, HL60, and K562 cells. Nonlimiting examples of colon cancer cells include SW48, SW480, SW116, CaCo-2, DLD1, Colo320, Colo205, HT29, and HT116 cells.

A noncancerous cell used in the methods can be any cancerous cell, and can be, as nonlimiting examples, a HEK293 cell, a COS-7 cell, a CHO cell, a NIH/3T3 cell, or a noncancerous colon cell, noncancerous intestinal epithelial cell, epithelial cell, skin cell, B cell, pre-B cell, T cell, pre-T cell, breast cell, prostate cell, liver cell, lung cell, ovarian cell, or cervical cell. Noncancerous colon (intestinal epithelial) cells include, without limitation, NCM356 cells and NCM460 cells ((Stauffer et al., Amer. J. Surg. 169: 190-195 (1995); Battacharya et al., Amer. J. Gastr. Liv. Physiol. 293: G429-437 (2007); both available from Incell Corp.), and NCIEM cells (Baten et al., FASEB J. 6: 2726 (1992)). Noncancerous cells can be transformed with the T antigen of SV40 to improve their transfectability. Primary cells can be isolated and immortalized by stably transfecting the cells with the T antigen of SV40 or hTERT (WO 2003/010305).

### Reporter Genes

Reporter genes include any genes whose expression is detectable, for example, by detection of the protein itself (e.g., fluorescent proteins), affinity-based detection of a domain of the protein (e.g., a peptide tag such as a flag tag or by expression of a peptide sequence that is a “self-labeling tag”, e.g., a FlASH or “lumio” tag that binds a fluorescent reagent) or by detecting the product of an enzymatic reaction catalyzed by the reporter protein

Fluorescent proteins include, without limitation, phycoerythrin, phycocyanin, allophycocyanin, a green fluorescent protein, a yellow fluorescent protein, a red fluorescent protein, an orange fluorescent protein, a cyan fluorescent protein, or a blue fluorescent protein. The variety of fluorescent proteins with different excitation and emissions spectra make them particularly useful where two or more reporter genes are desirable. Lentiviral vectors designed to investigate the expression of several genes in parallel in a single cell have been used to introduce three differently detectable fluorescent proteins in separate viral constructs into the same cell (Weber et al. Mol Ther. 16: 698-706 (2008)). Fluorescent protein detection is non-invasive, and may be done repeatedly on a same sample over time. Fluorescent protein genes used in the methods of the invention can be mutant forms of fluorescent protein genes. For example, the fluorescent protein genes can be mutants that are humanized or have enhanced fluorescence with respect to wild type proteins, or can be mutants with a higher turnover such that reporter gene measurements more accurately reflect a dynamic process such as changes in splicing or gene expression patterns in response to a modulating compound.

Enzymes that convert substrates to detectable products include alkaline phosphatase, beta galactosidase, beta lactamase, and luciferases. For example, substrates of alkaline phosphatase, beta galactosidase, beta lactamase can be conjugates that produce fluorescent compounds when cleaved. In some embodiments, secreted forms of these enzymes may be used.

Luciferases that can be used in the methods of the invention include, without limitation, beetle luciferases (including click beetle and firefly luciferases), Renilla luciferase, and Gaussia luciferase (Verhaegeb et al. Anal. Chem. 74: 4378-4385 (2002); Tannous et al. Mol. Ther. 11: 435-443 (2005)). Luciferase assays are quantitative and exhibit very low background. With the exception of the secreted Gaussia luciferase, luciferase assays generally require lysis of the assay cell. In some embodiments, however, a membrane-permeable luciferase reagent may be used, obviating cell lysis. Luciferases having different emissions optima can be used in two-reporter gene assays. For example, firefly luciferase and Renilla luciferase have distinguishable signals, and assay buffers are available that allow the signal from the two luciferases to be read in tandem (Promega Corp., Madison, Wis.). Click beetle red and green luciferase mutants have also been designed to have distinct emission spectra, so that two click beetle luciferase reporter genes can be used in the same assay using the same substrate buffer (Promega Corp., Madison, Wis.).

### Wnt Modulators and Activators

In some embodiments, noncancerous or cancerous cells used in the methods of the invention also include a recombinant construct that includes a gene for a Wnt activator or a Wnt modulator. A Wnt activator is any protein that when expressed in the cell, modulates Wnt signaling. Nonlimiting examples of Wnt activators include β-catenin, APC, axin1, axin2, GSK3, Disheveled, LRP5, LRP6, Frizzled, or Wnt proteins. A Wnt modulator is any protein that when expressed in the cell, modulates Wnt signaling by regulating the expression of one or more Wnt activators or one or more Wnt modulators. Nonlimiting examples of Wnt modulators include β-catenin, TCF-1, TCF-2, TCF-3, TCF-4, as well as the transcriptional repressors that interact with TCF/LEF proteins or β-catenin, including: CtBP, Groucho, Pygo, p300, and PIX2. In some embodiments, a Wnt activator or modulator expressed in cells is a mutant form of the activator or modulator. In some embodiments, the Wnt activator is a mutant APC gene. In some embodiments, the Wnt activator is a mutant β-catenin gene.

### Gene Transfer and Vectors

The recombinant reporter gene constructs or constructs for expression of Wnt modulators or activators that are used in the assay methods can be transiently transfected into cells, or can be integrated into the host cell. For transient transfection or selection of stable integrants, recombinant reporter gene constructs are preferably introduced into cells as plasmids. Nucleic acid constructs can be transfected into cells using any methods for introducing DNA into cells, including, for example, electroporation, DNA biolistics, lipid-mediated transfection, compacted bNA-mediated transfection, liposomes, dextran, immunoliposomes, lipofectin, cationic agent-mediated transfection, cationic facial amphiphiles (CFAs) (Nature Biotechnology 1996 14; 556), multivalent cations such as spermine, cationic lipids or polylysine, 1,2,-bis (oleoyloxy)-3-(trimethylammonio)propane (DOTAP)-cholesterol complexes (Woff and Trubetskoy 1998 Nature Biotechnology 16: 421) and combinations thereof. Selection of stable integrants is typically by selection on media containing an antibiotic for which the plasmid that includes the reporter gene construct has a resistance gene.

In some preferred embodiments of the invention, the reporter gene constructs or Wnt activator or Wnt modulator constructs are introduced into the cell using viral vectors or delivery systems. For example, the nucleic acid constructs can be introduced into cells using adenoviral vectors, adeno-associated viral (AAV) vectors, herpes viral vectors, or retroviral vectors (including lentiviral vectors). Viral vectors and delivery system provide the advantages of stable integration, the ability to transfect cells that may be otherwise recalcitrant to gene delivery methods, and single site integration of recombinant genes, providing a more reliable and consistent assay system. Inducible viral expression vectors include, for example, those disclosed in U.S. Pat. No. 6,953,575.

Retroviruses that can be used to reporter gene constructs and Wnt activator or modulator genes into cells include, without limitation: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and lentiviruses, which have the ability to infect both dividing and non-dividing cells.

Examples of primate lentiviruses include the human immunodeficiency virus (HIV), and simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

More than one retrovirus (or lentivirus) can be used to infect the same cell, providing the possibility of using retroviral vectors for introducing more than one reporter gene construct, Wnt modulator gene, Wnt activator gene, and combinations thereof. Infection of cells with three retroviruses can be done simultaneously, by infecting the cells with a mixture of the different engineered viruses, and selecting for cells carrying each of them (Weber et al. Mol Ther. 16: 698-706 (2008)).

### Test Compounds

Test compounds can be small molecules, peptides, polypeptides, carbohydrates, lipids, or nucleic acid molecules. A test compound can be a member of a library of natural or synthetic compounds. For example, test compounds can be from a combinatorial library, i.e., a collection of diverse chemical compounds generated by either chemical synthesis or biological synthesis by combining a number of chemical building blocks.

Test compounds can also include polypeptides and peptides, including peptide mimetics based on polypeptides. Test compounds can also be nucleic acid aptmers, nucleic acid molecule “decoys” of transcriptional promoter or enhancer sequences or splicing junctions or enhancers. In some embodiments, test compounds can be in the form of nucleic acid constructs that induce triple helical structures to inhibit transcription of a gene (Helene (1991) Anticancer Drug Des. 6:569-584).

In some embodiments, test compounds can include RNAi constructs or antisense oligonucleotides directed against one or more isoforms of a Wnt activator or modulator. In some embodiments, a test compound is a nucleic acid molecule that comprises one or more ribozymes directed against one or more isoforms of genes that partipate in Wnt signaling. The design, synthesis, and use of RNAi constructs, antisense oligonucleotides, and ribozymes are found, for example, in Dykxhoorn et al. (2003) Nat. Rev. Mol. Cell. Biol. 4: 457-467; Hannon et al. (2004) Nature 431: 371-378; Sarver et al. (1990) Science 247:1222-1225; Been et al. (1986) Cell 47:207-216).

For example, a test compound in some embodiments is an siRNA (“short interfering RNA”) molecule or a nucleic acid construct that produces an siRNA molecule. In some embodiments, test compounds are introduced into the cells as one or more short hairpin RNAs (“shRNAs”) or as one or more DNA constructs that are transcribed to produce one or more shRNAs, in which the shRNAs are processed within the cell to produce one or more siRNA molecules.

Nucleic acid constructs for the expression of siRNA, shRNA, antisense RNA, ribozymes, or nucleic acids for generating triple helical structures are optionally introduced as RNA molecules or as recombinant DNA constructs. DNA constructs for reducing gene expression or splicing of particular isoforms are optionally designed so that the desired RNA molecules are expressed in the cell from a promoter that is transcriptionally active in mammalian cells. For some purposes, it is desirable to use viral or plasmid-based nucleic acid constructs to introduce the test compounds.

### Pharmaceutical Compositions and Methods of Administration

Pharmaceutical compositions are formulated using one or more physiologically acceptable carriers including excipients and auxiliaries which facilitate processing of the active compounds into preparations which are used pharmaceutically. Proper formulation is dependent upon the route of administration chosen. A summary of pharmaceutical compositions is found, for example, in Remington: The Science and Practice of Pharmacy, Nineteenth Ed (Ea hston, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include one or more compounds that modulates of transcription or splicing of a Wnt isoform (a “Wnt isoform expression modulator”) or one or more antibodies that specifically binds an isoform of a protein that participates in Wnt signaling (“an isoform antibody”) and a pharmaceutically acceptable diluent(s), excipient(s), or carrier(s). In addition, the Wnt isoform expression modulator or isoform antibody is optionally administered as pharmaceutical compositions in which it is mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of a Wnt isoform expression modulator or isoform antibody with other chemical components, such as carriers, stabilizers, diluents, dispersing agents, suspending agents, thickening agents, and/or excipients. The pharmaceutical composition facilitates administration of the Wnt isoform expression modulator to an organism. In practicing the methods of treatment or use provided herein, therapeutically effective amounts of a Wnt isoform expression modulator or isoform antibody are administered in a pharmaceutical composition to a mammal having a condition, disease, or disorder to be treated. In some embodiments, the disease is cancer. Preferably, the mammal is a human. A therapeutically effective amount varies depending on the severity and stage of the condition, the age and relative health of the subject, the potency of the Wnt isoform expression modulator or isoform antibody used and other factors. The Wnt isoform expression modulator or isoform antibody is optionally used singly or in combination with one or more therapeutic agents as components of mixtures.

The pharmaceutical formulations described herein are optionally administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions in some embodiments will include at least one Wnt isoform expression modulator, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these Wnt isoform expression modulator having the same type of activity. In some situations, Wnt isoform expression modulators exist as tautomers.

“Carrier materials” include any commonly used excipients in pharmaceutics and should be selected on the basis of compatibility with compounds disclosed herein, such as, a Wnt isoform expression modulator, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

The pharmaceutical compositions described herein, which include a Wnt isoform expression modulator or isoform antibody, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

For administration by inhalation, the Wnt isoform expression modulator or isoform antibody is optionally in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of the Wnt isoform expression modulator and a suitable powder base such as lactose or starch.

Transdermal formulations of a Wnt isoform expression modulator or isoform antibody are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

Formulations that include a Wnt isoform expression modulator or isoform antibody suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, a Wnt isoform expression modulator or isoform antibody is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of the Wnt isoform expression modulator or isoform antibody in water soluble form. Additionally, suspensions of the Wnt isoform expression modulator or isoform antibody are optionally prepared as appropriate oily injection suspensions.

In some embodiments, the Wnt isoform expression modulator or isoform antibody is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

The Wnt isoform expression modulator or isoform antibody is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like.

### Splicing Assays

In some aspects, the methods include screening for compounds that affect RNA splicing of genes that affect cancer or participate in the Wnt signaling pathway. The test compounds can be screened for their ability to promote splicing, inhibit splicing, or alter the ratio of a first splice variant of a gene to a second splice variant of the same gene.

RNA splicing can be detected by any of a variety of assays that detect the presence or absence of an exon in an RNA, including, without limitation, detection of protein domains encoded by particular exons (or introduced into particular exons) in translated proteins, electrophoretic separation and gel analysis of RNA or protein, polymerase chain reaction-based assays, Northern analysis or RNase protection using exon-specific probes, invasion cleavage assay (Eis et al. Nature Biotechnol. 19: 673-676 (2001), radionucleotide or fluorescently labeled nucleotide incorporation, etc. In some embodiments, splicing assays are performed using reporter gene assays.

Reporter gene assays that can be used to detect splicing, include, without limitation, assays in which production of an active reporter gene requires splicing out of an intron within the reporter gene; assays in which production of an active reporter gene requires splicing in of an intron within the reporter gene; assays in which splicing efficiency is measured using a two reporter gene construct, in which production of both active reporter gene open reading frames requires splicing out of an intron that is positioned between the reporter genes (Nasim et al. Nature Protocols 1: 1022-1028 (2006)), and two reporter gene assays in which the reading frame of one or the other of the reporters is shifted depending on the alternative splicing event that occurs (Nasim et al. Nucl. Acids Res. 30: 109-125 (2002); Newman et al. RNA 12: 1129-1141 (2006); Orengo et al. Nucl. Acids Res. 34: 148-154 (2006)).

A gene having splice variant associated with cancer is a gene in which the relative proportions of isoforms are different in cancer cells than in normal cells of the same type. In some embodiments, a particular isoform of a protein is reduced in amount or proportion to another isoform of the protein in cancerous cells with respect to normal cells of the same type. Detection of amount or relative proportions of isoforms can be detection of protein or RNA level or amount. In some embodiments, a particular isoform is not detectable in cancerous cells but is present in normal cells of the same type.

Exons to be tested for inclusion (“splicing in”) or exclusion (“splicing out”) from an RNA transcribed from a gene of interest can be identified for example, using microarrays (Xiao et al. PLoS Compoutational Biology 1: 276-288 (2005)) and/or published databases (for example, genome.ewha.ac.kr/ECgene). Such methods can be used to identify exons that are alternatively expressed in disease tissues with respect to equivalent normal tissue. For example, the expressed gene sequences of cancer cells and normal cells of the same cell type can be compared to identify exons that are preferentially present or absent in processed RNAs of cancer cells with respect to normal cells. The genomic intronic sequences surrounding alternatively spliced exons can be compared and priority weighted to identify sequences that affect splicing; this information can be used to optimize the sequence of a reporter gene for use in screens, such that the optimized reporter gene does not affect splicing. Test compounds can be assessed for their ability to affect splicing of a gene having isoforms whose levels or proportions to other splice variants are altered in cancer cells with respect to normal cells.

For example, for assays that test for compounds that restore expression of an alternatively spliced exon, an optimized reporter gene can then be inserted, in frame, near the 3′ end and 5′ of the splice donor site of an exon whose expression is reduced in disease state cells. The expression of this reporter gene in assays is indicative of restoration of expression of the exon in the assayed cells: increased expression of the reporter gene indicates an increased level of transcript in which the alternatively spliced exon is “spliced in” to the RNA that is translated in the cells.

Many spliced genes give rise to alternatively spliced transcripts whose relative proportions may change in a disease state. To test for compounds that can shift the ratio of one alternative splicing isoform (for example, a splicing isoform that increases in prevalence in a disease state) to a second splicing isoform, dual reporter gene constructs are useful, where the ratio of expression of a first reporter gene to a second reporter gene reflects the ratio of occurrence of a first splicing event to a second splicing event.

A method is provided herein for identifying a compound that modulates alternative splicing of a gene having splice variants associated with cancer. The method includes: providing a cell that includes a nucleic acid construct, in which the nucleic acid construct comprises a transcription unit having a promoter, a first reporter gene and a second reporter gene, in which the first reporter gene and the second reporter gene are differently detectable, and an alternative splice module that has three exons, in which the sequences of the exon-intron boundaries of the alternative splice module are derived from a gene that is alternatively spliced, in which a splice variants of the gene is associated with cancer. The splice module is configured such that a first alternative splicing event results in the splicing of the first exon to the second exon and the splicing of the second exon to the third exon, resulting in the expression of the first reporter gene and, and the second splicing event results in the splicing of the first exon to the third exon, resulting in the expression of the second reporter gene. The method includes contacting the cell with a test compound, detecting the signals from expression of the first reporter gene and the second reporter gene, calculating a ratio of the expression of the first reporter gene to the second reporter gene, and detecting a difference between the reporter gene expression ratio in the cell contacted with the test compound to the reporter gene expression ratio in a cell not contacted with the test compound, where a difference in the reporter gene expression ratio indicates that the test compound modulates a cancer-associated alternative splicing process.

A “cancer-associated alternative splicing process” is a splicing process that results in production of a cancer-associated splice variant. The term “cancer-associated splice variant” refers to a splice variant that is more abundant or has a higher relative abundance in cancer cells when compared with noncancerous cells of the same type. A “higher relative abundance” means a higher abundance relative to an alternative splice variant of the same gene. A cancer-associated splice variant can also refer to a splice variant that participates in a signaling pathway that is associated with the cancerous state. In some embodiments of the invention, a cancer associated splice variant is a splice variant of Ron.

In some embodiments of the invention, a cancer associated splice variant is a splice variant that participates in the Wnt signaling pathway. In some embodiments of the invention, a cancer associated splice variant is a splice variant of a Wnt activator, Wnt modulator, or Wnt target gene. Nonlimiting examples of wnt activators and wnt modulators that are alternatively spliced are LRP5/LRP6 (BMC Genomics 7: 148 (2006)), axin-1 (BMC Genomics 7: 148 (2006)), beta-catenin (Roth et al. Genes chrom Cancer 44: 423-428 (2005); Pospisil et al. BMC Genomics 7: 148 (2006)), axin-2 (Hughes et al. J. Biol. Chem. 280:8581-8588 (2005)), APC (Hori et al. Hum Mol Genet. 2: 283-287 (1993)), GSK3-beta (BMC Genomics 7: 148 (2006)); TCF-1 (J. Biol. Chem. 267: 8530-8536 (1992); Mol. Cell. Biol. 16: 745-752 (1996)), TCF-3, TCF-4 (J. Biol. Chem. 278: 16169-16175 (2003)); LEF-1 (Nucl. Acids Res. 28: 1994-2003 (2000); Devel. Dynamics 232: 969-978 (2005)), and CtBP1 (BMC Genomics 7: 148 (2006)). Any of these genes can be investigated for the association of any of their splice variants with cancer. An alternative splice module of an alternative splice assay construct can include exons derived from exons of any of these genes to identify compounds that affect alternative splicing of genes that encode proteins that participate in wnt signaling.

In some embodiments of the invention, a cancer associated splice variant is a splice variant of Bcl-X or a TCF/LEF protein. In some embodiments, a gene construct that includes at least a portion of an exon of the TCF-1, TCF-4, LEF-1, or Bcl-X gene is used in assays to identify compounds that affect alternative splicing of a TCF-1, TCF-4, LEF-1, or Bcl-X RNA transcript.

Bcl-X is a Wnt target gene (J. Cell Biol. 176: 929-939 (2007); (Cancer Research 61: 6876-6884 (2001); J. Biol. Chem. 276: 21062-21069 (2001)), and Bcl-X(S) (short isoform) is a dominant negative repressor of Bcl-2 and Bcl-X(L). Expression of Bcl-XS reduces tumor size (Ealovega et al., 1996) and sensitizes tumor cells to chemotherapeutic agents (Sumatran et al., 1995). In one embodiment the splice module is derived from at least exons 1, 2, and 3, of the Bcl-X gene, in which alternative splicing of Bcl-X exon 3, gives rise to pro-apoptotic (Bcl-x(S)) and anti-apoptotic (Bcl-x(L)) proteins. In another example, the splice module is derived from at least exons VIII, IX, and X of the LEF1 gene, in which alternative splicing of exon IX gives rise to LEF-1B (van de Wetering et al. Mol. Cell. Biol. 16: 745-753 (1996)). In another example, In another example, the splice module is derived from at least exons 10, 11, and 12 of the TCF-1 gene, in which alternative splicing of exon 11 results in the splice variant TCF-1E (Hovanes et al. Nucl. Acids Res. 28: 1994-2003 (2000). In another example, the splice module is derived from at least exons 7, 8, and 9 of the TCF-4 gene, in which alternative splicing of exon 8 results in the splice variant TCF-4E (Hovanes et al. Nucl. Acids Res. 28: 1994-2003 (2000).

In some embodiments, the alternative splice module is configured such that when the alternative splice construct is transcribed, the following two alternative splicing events can occur: A first alternative splicing results in the splicing of the first exon to the second exon, which is spliced to the third exon, resulting in the expression of a first reporter gene and the second reporter gene. A second alternative splicing event results in the splicing of the first exon to the third exon, resulting in the expression of the second reporter gene but not the first reporter gene. The method includes contacting the cell having the alternative splicing construct with a test compound, detecting the a signal from expression of the first reporter gene and a signal from expression of the second reporter gene, and calculating a ratio of the expression of the first reporter gene to the second reporter gene. The difference between the first and second reporter gene expression ratio in the cell contacted with the test compound to the first and second reporter gene expression ratio in a cell not contacted with the test compound are compared, and a difference in the reporter gene expression ratio of test compound-contacted cells with respect to control cells identifies the test compound as a compound that modulates cancer-associated alternative splicing process.

In these embodiments of the method, the first reporter gene is embedded in-frame within exon 2 of the alternative splicing construct, and the second reporter gene is embedded in-frame within exon 3 of the alternative splicing construct. In these embodiments, a splicing event that joins exons 1, 2, and 3 results in expression of a two reporter gene protein, in which both reporter genes give a detectable signal. The reporter genes can be any reporter genes that have distinguishable signals, for example, two fluorescent protein with different emissions wavelengths, two luciferases with different emissions wavelengths, a luciferase and a fluorescent protein (with distinguishable emissions wavelengths), a luciferase and beta-galactosidase, a luciferase and beta-lactamase, a luciferase and an alkaline phosphatase, etc.

The splicing assay constructs in some embodiments can include more than three exons, for example, the splicing assay constructs can include 4, 5, 6, or more exons, in which the intron/exon boundaries of the exons are derived from a gene that encodes a protein that participates in Wnt signaling. In some embodiments of the methods, a splicing assay construct includes an alternative splice module that includes 4, 5, 6, or more exons, in which the intron/exon boundaries of the exons are derived from a gene that encodes a protein that participates in Wnt signaling, and a reporter gene is embedded in each of the exons of the alternative splice module. In preferred embodiments, at least two of the reporter genes of the splice module are differently detectable. In some preferred embodiments, all of the reporter genes of the splice module are differently detectable.

In other embodiments of the method, the alternative splice module is configured such that the first reporter gene and the second reporter gene are both inserted in tandem into exon 3, or at the 3′ end of exon 3. A base insertion or deletion is made in exon 2, such that when exon 2 is included in the splice product, a reading frame shift occurs. Any stop codons in exon 2 generated by this insertion are mutated to non-stop codons, as are any stop codons in the shifted reading frame of the first reporter gene. In these embodiments, a first splicing event results in expression of a first reporter protein (and not a second reporter protein), and a second splicing event results in expression of a second reporter protein (and not a first reporter protein) due to a difference in reading frame of the two reporter proteins. The first splicing event that joins exons 1, 2, and 3 results in expression of a protein in which the first reporter gene is out-of-frame (although lacking stop codons, so that there is translation through the sequence into the second reporter gene), and the second reporter gene is in-frame, producing a detectable signal. The second splicing event that joins exons 1 and 3 results in expression of a protein in which the first reporter gene is in-frame, producing a detectable signal, and the second reporter gene is out-of-frame, producing no signal.

Any two reporter genes that have distinguishable signals, for example, two fluorescent protein with different emissions wavelengths, two luciferases with different emissions wavelengths, a luciferase and a fluorescent protein (with distinguishable emissions wavelengths), a luciferase and beta-galactosidase, a luciferase and beta-lactamase, a luciferase and an alkaline phosphatase, etc. can be used in these methods. In embodiments in which both reporter genes are embedded in or appended to exon 3, in which splicing of an exon causes a reading frame shift that determines which of two downstream reporter genes will be expressed in-frame, the first reporter gene must have, or be mutated to have, no stop codons in one of its alternate reading frames. In these embodiments, read-through can occur through the first reporter gene (which will be translated in a reading frame other than its proper fluorescent protein encoding reading frame) to the open reading frame of the second reporter gene when the two reporter genes are configured in tandem. The red fluorescent protein Ds red, which has a +1 (non Ds red-encoding) reading frame with no stop codons, is particularly useful as the first reporter gene, which can be read through to create a fusion protein that includes an active second reporter protein domain (Orengo et al. Nucl. Acids Res. 34: 148-154 (2006); Newman et al. RNA 12: 1129-1141 (2006)). The second reporter gene can be for example, GFP, a luciferase, beta-galactosidase, beta-lactamase, or alkaline phosphatase gene.

Compounds tested using the assay methods provided herein can be any compounds, including, without limitation, small molecules, peptides, proteins, and nucleic acids or combinations thereof. In some embodiments, pladienolide and derivatives thereof are tested using the assays methods provided herein for assaying alternative splicing frequency of exons identified as being alternatively spliced in a disease state in genes of interest (Nature Chem. Biol. 3: 570-575 (2007)). In some embodiments, NB-506 and derivatives thereof are tested using the assays methods provided herein for assaying alternative splicing frequency of exons identified as being alternatively spliced in a disease state in genes of interest (Cancer Research 61: 6876-6884 (2001)).

Test compounds identified as compounds that reduce the frequency of inclusion of an alternatively spliced exon, where increased expression of the splice variant that includes the alternatively spliced exon is indicative of a disease state, or test compounds that increase the frequency of inclusion of an alternatively spliced exon, where reduced expression of the splice variant that includes the alternatively spliced exon is indicative of a disease state, can be tested for their ability to affect one or more additional properties of the cell that are characteristic of the disease state. Such properties can include, without limitation, cell growth rates, metabolic status, motility, migration, invasiveness, or adhesion, or the expression of particular genes or proteins by the cell. The invention also includes, in these aspects, methods for identifying compounds that affect the behavior of disease state cells by identifying compounds that affect alternative splicing in disease state cells. The invention also includes, in further aspects, methods for treating a disease state by administering to a subject diagnosed with a disease one or more compounds that affect alternative splicing in disease state cells. In some embodiments, the disease state is cancer.

### LEF-1 P2 Promoter Assay

In another aspect of the invention, test compounds are screened for their ability to upregulate the P2 promoter of the LEF-1 gene, which directs transcription of the dN isoform of LEF-1. The dN (“dominant negative”) isoform of LEF-1 lacks the beta catenin domain of the full length (FL) isoform of LEF-1 and therefore may act as a dominant negative to suppress Wnt signaling.

Provided herein is a method for identifying a compound that promotes transcription of the dominant negative form of LEF1, in which the method includes: providing a cell that comprises a reporter gene regulated by the P2 promoter of the LEF-1 gene, contacting the cell with a test compound, and detecting an increase in the signal from expression of the reporter gene in the cell contacted with the test compound as compared with the expression of the reporter gene in a control cell not contacted with the test compound to identify a compound that upregulates transcription of the dominant negative form of LEF1.

The P2 promoter is any subset of the sequence of the LEF-1 gene from about −4000 to +100, where +1 corresponds to the P2 transcriptional start site, and is preferably a subset of the sequence of the LEF-1 gene that comprises a repressor region, between bases −1446 and −1281 (Li et al., Mol. Cell. Biol. 26: 284-5299) as well as the basal P2 promoter region between bases −177 and +60. The LEF-1 promoter can include sequences of the LEF-1 gene from about −5000 to about +100, or about −4000 to about +100, or about −3000 to about +100, or about −2000 to about +60, or about −1500 to about +60, or about −1450 to about +60, in which +1 is the start site of the P2 promoter, which is 10 bases upstream of the 5′ end of exon 3. In some aspects of the invention, the cell in which the assay is performed is a cancerous cell, such as, for example a colon cancer cell, which can be, as nonlimiting examples, a Colo320 cell, a DLD1 cell, an SW480 cell, or an HT29 cell.

In some aspects of the invention, the cell is a cancerous or noncancerous that also includes a nucleic acid molecule construct that encodes a Wnt activator or regulator under the control of a constitutive or inducible promoter. In some embodiments, the cell is a normal colon cell, such as, for example, NCIEM, NCM460, or NCM356 cells that constitutively or inducibly express a mutant form of beta catenin or a mutant APC gene, such that when the Wnt activator or regulator is expressed, the wnt pathway is activated in the cells.

In some embodiments, assays to screen compounds for the ability to upregulate the LEF-1 P2 promoter are performed using colon cancer cells and normal colon cells, or using normal colon cells expressing a Wnt activator or modulator, such that the Wnt pathway is activated, and normal colon cells not expressing a Wnt activator or modulator. In these embodiments, a compound that demonstrates upregulation of the LEF-1 P2 promoter in cancerous cells, or normal cells expressing a Wnt activator or modulator, but does not significantly upregulate the LEF-1 P2 promoter in normal cells that do not express an introduced wnt activator or modulator, is identified as compound having specificity for upregulating the P2 promoter.

In some embodiments, the assay cells include a constitutively regulated reporter gene as an internal control. The signal form the reporter gene linked to the LEF-1 P2 promoter is normalized to the signal detected from the reporter protein that is not regulated by a P2 promoter. Where noncancerous cells not expressing a Wnt modulator or activator are also assayed for the response of a P2 reporter construct to a test compound, the noncancerous cells also preferably have a reporter gene under the control of a constitutive promoter as a control.

### Dual Promoter Assay

In another aspect of the invention, a method for identifying a compound that affects the expression of an isoform of a protein that participates in Wnt signaling is provided, in which the method includes: providing a cell that comprises a dual promoter reporter gene construct, in which the dual promoter construct has a promoter region of a gene that has two promoters, in which a different isoform of the gene is transcribed from each of the two alternative promoters. In these embodiments, the promoter assays replicate the proximity of alternative promoters in the cell, such as for example the LEF1 gene, which has two transcriptional start sites within 5.5 kb of one another, where activation of the P1 promoter may affect activation at the P2 promoter, and vice versa.

The dual promoter construct includes two differently detectable reporter genes operably linked to the dual promoter region of the gene that affects Wnt signaling, and is configured such that expression of the first reporter gene results from transcription from the first alternative promoter and expression of the second reporter gene results from transcription from the second alternative promoter of the dual promoter region. The cell having the dual promoter reporter gene construct is contacted with a test compound, and the signal from expression of the first reporter gene and the second reporter gene is detected. A test compound that changes the ratio of expression of the first reporter gene to expression of the second reporter gene with respect to the ratio of expression of the first and second reporter genes in cells that are not contacted with the test compound, is identified as a compound that affects expression of a transcriptional isoform of a gene.

Test compounds identified as compounds that affect the expression of a transcriptional isoform of a gene can be tested for their ability to affect one or more additional properties of the cell that are characteristic of the disease state. Such properties can include, without limitation, cell growth rates, viability/cytotoxicity, metabolic status, apoptosis, motility, migration, invasiveness, or adhesion, or the expression of particular genes or proteins by the cell. The invention also includes, in these aspects, methods for identifying compounds that affect the behavior of disease state cells by identifying compounds that affect alternative promoter use in disease state cells. The invention also includes, in further aspects, methods for treating a disease state by administering to a subject diagnosed with a disease one or more compounds that affect alternative promoter use in disease state cells. In some embodiments, the disease state is cancer.

In some preferred embodiments, at least one of the transcriptional isoforms of the gene is related to cancer, in which one of the isoforms is present at a greater or lesser amount in a cancer cell as compared to a normal cell of the same type. In some preferred embodiments, the gene encodes a protein that participates in Wnt signaling. In some embodiments, the gene is LEF1, TCF1, or Bcl-X.

For example, the region of the LEF-1 gene extending upstream to at least −64 (where +1 is the transcriptional start site from the P1 promoter; Hovanes et al. Nucl. Acids Res. 28: 1994-2003 (2000)) and extending downstream to at least 50 nucleotides into exon 3, can be used as a promoter region in a dual promoter construct. This sequence, which includes both the P1 and P2 promoters, extends from 64 nucleotides upstream of the “full length” transcriptional start site upstream of exon 1, through exon 1, intron 1, exon 2, intron 2, and approximately 50 nucleotides into exon 3. In some preferred embodiments, the promoter region includes sequences further upstream of the P1 promoter, extending from approximately 670 nucleotides upstream of the P1 transcriptional start site to approximately 50 nucleotides into exon 3.

In embodiments in which the LEF-1 dual promoter region is used in the alternative promoter construct, the P1 promoter initiates transcription at exon 1, and the splice product of the P1 transcript includes exons 1, 2, and 3. The P2 promoter initiates transcription immediately upstream of exon 3, and the splice product of the P2 transcript includes exon 3 but does not include exons 1 and 2. Dual promoter constructs used in the methods for detection of the two transcripts have a reading frame shift introduced into exon 2, and have two reporter genes inserted in tandem into exon 3, such that when P1 is used as the promoter, the first reporter gene is translated in its proper reading frame, but the second reporter gene is out of frame, and when P2 is used as a promoter, the first reporter gene is transcribed in a reading frame that does not include stop codons but is not its proper reading frame for encoding the reporter protein, and the second reporter gene is expressed as a fusion protein in its proper reading frame. Thus, detection of a signal from the first reporter gene is indicative of transcription from P1, and detection of a signal from the second reporter gene is indicative of transcription from P2. The ratio of the P1 signal to the P2 signal represents the ratio of P1 transcription to P2 transcription.

### Cancer-Specific Isoforms

Also provided are methods for identifying a cancer-specific isoform sequence of a gene, in which the methods include: comparing RNA transcripts of genes or cDNA or amplified DNA generated from RNA transcripts of genes isolated from cancer cells and normal cells of the same cell type, and identifying one or more exons uniquely present in RNA transcripts or cDNA generated from RNA transcripts of the cancer cells to identify at least one cancer-specific isoform sequence of a protein.

Also provided are methods for identifying a cancer-specific isoform sequence of a Wnt-related gene, in which the methods include: comparing RNA transcripts of genes or cDNA or amplified DNA generated from RNA transcripts of genes isolated from cancer cells and normal cells of the same cell type, and identifying one or more exons uniquely present in RNA transcripts or cDNA generated from RNA transcripts of the cancer cells to identify at least one cancer-specific isoform sequence of a protein. Nonlimiting examples of wnt activators and wnt modulators that are alternatively spliced are LRP5/LRP6 (BMC Genomics 7: 148 (2006)), axin-1 (BMC Genomics 7: 148 (2006)), beta-catenin (Roth et al. Genes chrom Cancer 44: 423-428 (2005); Pospisil et al. BMC Genomics 7: 148 (2006)), axin-2, APC (Hori et al. Hum Mol Genet. 2: 283-287 (1993)), GSK3-beta (BMC Genomics 7: 148 (2006)); TCF-1 (J. Biol. Chem. 267: 8530-8536 (1992); Mol. Cell. Biol. 16: 745-752 (1996)), TCF-3, TCF-4 (J. Biol. Chem. 278: 16169-16175 (2003)); LEF-1 (Nucl. Acids Res. 28: 1994-2003 (2000); Devel. Dynamics 232: 969-978 (2005)), and CtBP1 (BMC Genomics 7: 148 (2006)).

In some embodiments, the RNA transcripts are compared by comparing databases of expressed genes or expressed sequence tags (ESTs) (Xu et al. Nucl. Acids Res. 31: 5635-5643 (2003)). In some embodiments cancer associated isoforms are identified using microarrays (Xiao et al., PLoS Compoutational Biology 1: 276-288).

Also included is a method for identifying a cancer-specific domain of a protein, such as a protein that participates in wnt signaling, that includes performing mass spectrometry on proteins isolated from cancer cells and on proteins isolated from normal cells of the same cell type, and identifying one or more protein sequences of one or more Wnt-related proteins uniquely present in the cancer cells to identify a cancer-specific sequence of a Wnt-related protein. For example, proteins of cancer cells can be metabolically labeled with heavy isotopes for comparison of their protein profile with the protein profile of normal cells of the same type, or normal cells can be heavy-isoptope labeled and their proteins can be compared using mass spectrometry with proteins isolated from cancer cells of the same type to identify splice variants or variants arising from alternative promoter use (U.S. Pat. No. 6,642,059).

An isoform-specific nucleic acid sequence or a portion thereof can be expressed in cells or in vitro, for example, as part of the cancer specific protein isoform, or as a fusion protein with other protein sequences, or on its own. Alternatively, a peptide having at least a portion of the isoform-specific sequence can be synthesized. Peptide or proteins that include at least a portion of the cancer associated isoform-specific protein sequence can be used to generate antibodies.

The invention further includes a method of obtaining an antibody specific to an isoform of a Wnt-related protein that is present in cancer cells but not present in normal cells, in which the method includes: identifying an amino acid sequence of a wnt-related protein isoform that is uniquely present in cancer cells, expressing or synthesizing the amino acid sequence, and generating an antibody to the amino acid sequence to obtain an antibody that binds to an isoform of a wnt-related protein that is present in cancer cells but not present in normal cells of the same type. In preferred embodiments the antibody recognizes the cancer-specific isoform and does not recognize isoforms of the protein that are not cancer-specific.

The invention also includes antibodies that specifically bind to an isoform of a protein that is present in cancer cells but not present in normal cells of the same type, in which the antibody does not specifically bind to a protein in normal cells of the same type. For example, an antibody can be specific to an isoform of a wnt-related protein that is present in cancer cells but not in normal cells. In some embodiments, the antibody specifically binds the E tail domain of TCF-4E.

The antibody can be a monoclonal antibody or a polyclonal antibody. As used herein, “antibody” can also mean an active fragment of an antibody, and includes Fab, Fab(2), single chain antibodies, chimeric antibodies, and humanized antibodies that can be made by modification of whole antibodies of by recombinant methods.

Antibodies specific to domains of proteins that are expressed in cancer cells but not in normal cells of the same type (“cancer-specific domains”) can be used for therapeutically or for diagnosis or imaging of cancer cells. For example, an antibody with specificity for a particular cancer-associated isoform of a protein can be an antibody that can inhibit a function of the protein, such as a catalytic function or a binding function. In some embodiments, an antibody can disrupt protein-protein interactions of a cancer-associated isoform of a protein. In some embodiments, an antibody can disrupt protein-protein interactions of an isoform of a protein that affects wnt signaling, such as, for example, a wnt activator or wnt modulator.

Antibodies for therapeutic use are in some embodiments coupled to or formulated with peptides or other reagents that facilitate entry of protein into the cells. Cell penetrating peptides such as the TAT protein of HIV, penetratin, transportan, and pVEC (Saalik et al. Bionconjug. Chem. 15: 1246-1253), the pHLIP peptide (Andreev et al. Proc Natl Acad Sci 104: 7893-7898 (2007); the YTA2 peptide (Myrberg et al. Bioconjug Chem 18: 170-174 (2007)); the SAINT-PHD™ delivery reagent (Synvolux Therapeutics); CHARIOT™ (Active Motif, Carlsbad, Calif.), and PROVECTIN™ protein delivery agent (Imgenex, San Diego, Calif.), are nonlimiting examples of such peptides and reagents for protein and peptide delivery.

In some embodiments, an antibody that specifically recognizes a cancer specific isoform of a protein, such as a cancer-specific isoform of a protein that participates in Wnt signaling, is coupled to a therapeutic or cytotoxic agent. For example, an antibody to a cancer-specific isoform of a protein can be conjugated to a small molecule toxin such as, but not limited to, calicheamicin or a structural analogue thereof (Hinman et al. Cancer Res. 53: 3336-3342 (1993); Lode et al. Cancer Res. 58: 2925-2928 (1998)); maytansine (U.S. Pat. No. 5,208,020), a trichothene, or CC1065. Other toxins to which an antibody can be conjugated include, without limitation, diptheria A chain, endotoxin A chain, ricin A chain, abrin A chain, modeccin, alpha-sarcin, dianthin proteins, Phytolaca americana proteins, momordica charantia inhibitor, curcin, crotin, sapaonaria officinalis inhibitor, gelonin, mitogellin, restrictocin, phenomycin, enomycin, and tricothecenes.

An antibody that specifically recognizes a cancer specific isoform of a protein, such as a cancer-specific isoform of a protein that participates in Wnt signaling, can also be coupled to a nuclease or a radioactive isotope such as, but not limited to, Y⁹⁰, At²¹¹, Re¹⁸⁶, Re¹⁸⁸, Sm¹⁵³, Bi²¹², P³², and radioisotopes of Lu.

Antibodies to cancer-specific isoforms can also be used diagnostically. In these aspects, an antibody that specifically binds a cancer-specific isoform of a protein, such as a protein that participates in Wnt signaling, can be used to detect one or more cancer cells in a sample, such as a sample from an individual. The antibody in some embodiments in bound to a soluble support, such as a filter, strip, membrane, well, chip, particle, or bead. The antibody in some embodiments is linked to a detectable label or enzyme.

Antibodies to cancer-specific isoforms can also conjugated to fluorophores or other imaging agents for detection of cancer cells or imaging of tumors. An imaging agent in some embodiments can be an isotope such as but not limited to: ¹⁸F, ⁴³K, ⁵²Fe, ⁵⁷Co, ⁶⁷Cu, ⁶⁷Ga, ⁷⁷Br, ⁸⁷MSr, ⁸⁶Y, ⁹⁰Y, ⁹⁹MTc ¹¹¹In, ¹²³I, ¹²⁵I, ¹³¹I, ¹³²I, ¹²⁷Cs, ¹²⁹Cs, ¹⁹⁷Hg, ²⁰³Pb, or ²⁰⁶Bi.

### Example 1

### Splicing Assay with Single v. Double Reporter Gene Expression

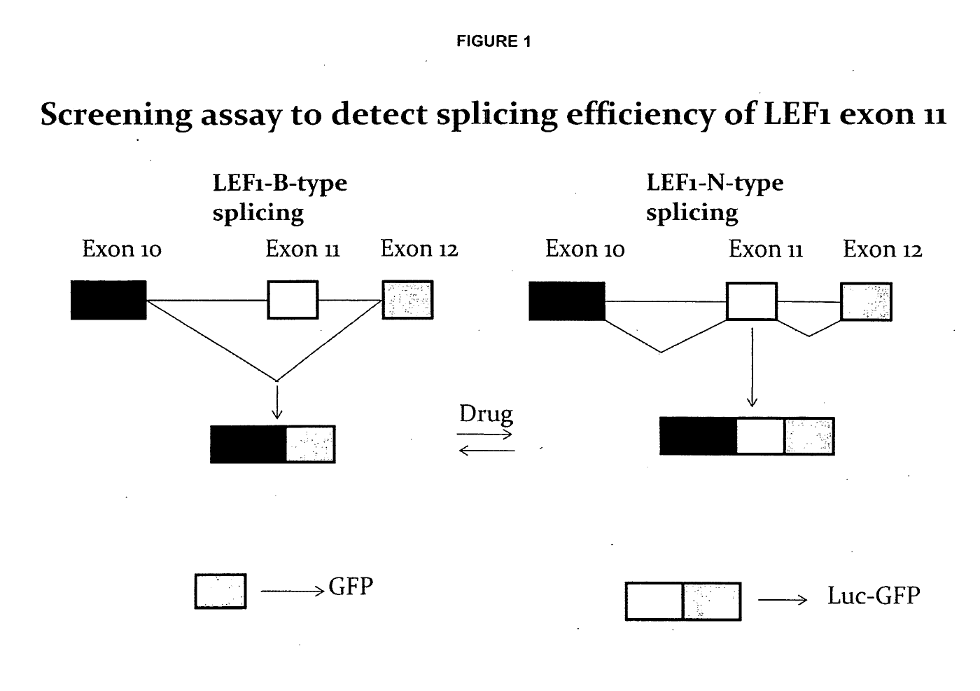

A reporter gene construct having the alternative splice module shown in FIG. 1 is transiently transfected into SW480 T cells. FIG. 1 shows a construct for a screening assay to detect splicing efficiency of LEF1 exon 11 The alternative splice module is derived from the LEF-1 gene, and has the region of the LEF-1 gene that includes exon 10, exon 11, and exon 12 of the LEF-1 gene, and the introns that separate them, including the sequences of the exon-intron boundaries, except that the central region of exon 11 has the luciferase gene inserted within it, replacing a portion of the open reading frame of exon 11, and the central region of the open reading frame of exon 12 has been replaced with a de-stabilized green fluorescent protein gene (available from Clonetech Inc., Mountain View, Calif.). The splice module is configured such that the luciferase gene is in frame with the open reading frame of exon 11 sequences and when exon 11 is spliced to exon 12, the open reading frame continues through in the proper reading frame for the green fluorescent protein gene that is inserted into exon 12.

When the alternative splice module is expressed in the cell, two alternative splicing events can occur. In the first, exon 10 splices to exon 11 with in turn splices to exon 12. In this case, both luciferase and green fluorescent protein coding sequences are present, in the correct reading frame, in the resulting spliced mRNA. In the second alternative splicing event, exon 10 splices to exon 12 (exon 11 is spliced out) and only the green fluorescent protein coding sequences are present, in the correct reading frame, in the resulting spliced mRNA.

Twenty-four hours after transfection with the alternative splice module, the cultured SW480/LuGSM cells are distributed at approximately 10,000 cells per well into 384 well multiwell plates. Test compounds from a compound library are added to the wells to a final concentration of 0.5 micromolar. A series of control wells for each cell type receive only buffer or solvent. After a further twenty-four hour incubation, luciferase buffer is added, and 10 minutes later the signal from luciferase is detected followed by detection of the signal from green fluorescent protein.

Wells to which a test compound has been added having readings that indicate an altered ratio of luciferase signal to green fluorescent protein signal on addition of test compound with respect to control wells that lack test compound are used to identify a test compound that modulates splicing of a Wnt-related gene (LEF-1).

### Example 2

### Splicing Assay with Alternative Reporter Gene Expression

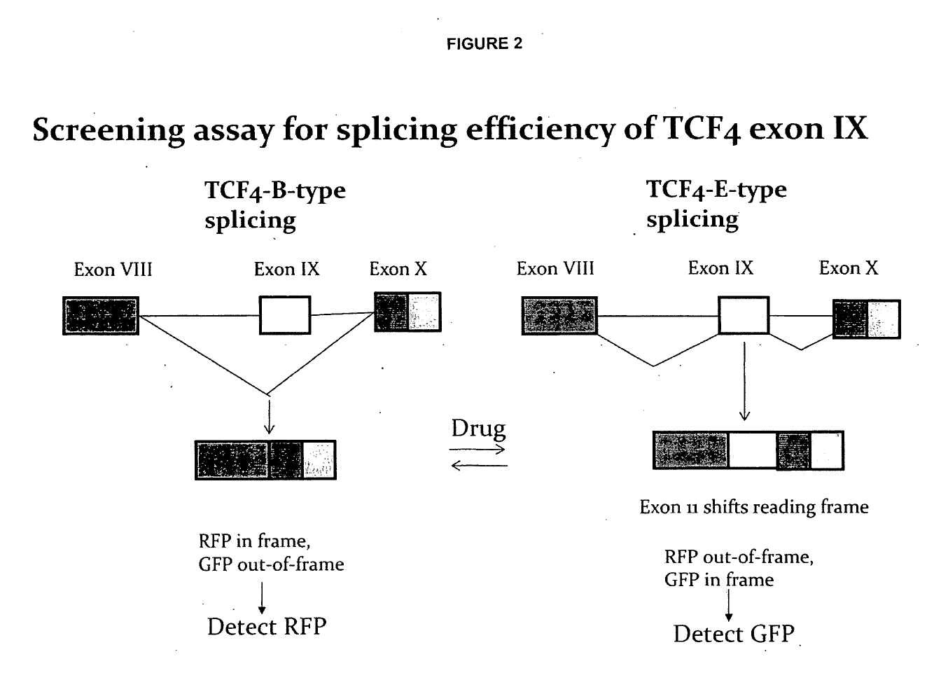

A reporter gene construct having an alternative splice module as shown in FIG. 2 (screening assay for splicing efficiency of TCF₄ exon IX) is introduced into SW480 cells via a lentivirus. The alternative splice module is derived from the TCF-4 gene, and has the region of the TCF-4 gene that includes exon 8, exon 9, and exon 10 of the TC4 gene, and the introns that separate them, including the sequences of the exon-intron boundaries, except that 3′ end of exon 10 has a red fluorescent protein gene and a green fluorescent protein gene tandemly appended to it. The splice module is configured such that the red fluorescent protein gene is in frame with the open reading frame of exon 8 sequences when exon 8 is spliced to exon 10, but is not in frame with the GFP gene. When exon 9 is spliced out, therefore, the red fluorescent protein is expressed but the green fluorescent protein is not in frame and is not expressed. When exon 9 is spliced in, the second (“+1”) open reading frame of the dsRed gene is in frame with the reading frame of exon 9 and the GFP gene in exon 10. However although the “+1” reading frame of dsRed does have a stop codon, it does not encode a fluorescent protein, so the result of exon 9 being spliced in is that only GFP is detectable.

The cultured SW480/splice reporter cells are suspended and distributed at approximately 10,000 cells per well into 384 well multiwell plates. Test compounds from a compound library are added to the wells to a final concentrations ranging from 10 picomolar to 10 micromolar. A series of control wells receive only buffer or solvent. The signal from red fluorescent protein and the signal from green fluorescent protein are detected 0, 4, 8, 16, and 24 hours after the addition of the compounds. SW480/splice reporter cells wells having readings that indicate a lower level of expression of luciferase and a higher level of green fluorescent protein on addition of test compound are identified as wells to which splicing modulators of a Wnt modulator gene have been added.

### Example 3

### P2 Promoter Assay

An assay is performed to identify a compound that upregulates expression from the P2 promoter of the LEF-1 gene.

A DNA construct comprising a region of the LEF1 gene that includes the P2 promoter is linked to a luciferase reporter gene. The region of the LEF1 gene included in the construct extends from −1500 to +60, where the +1 transcriptional start site is 10 bp upstream of the start of intron2/exon 3 border of the LEF1 gene. This region includes the repressor region of the promoter (Li et al. Mol. Cell. Biol. 26: 5284-5299). The −1500-+60 region of the LEF1 gene that includes the P2 promoter is operably linked to a CHROMA-LUC™ CBG68luc green light-emitting luciferase gene (Promega, Madison, Wis.). A CHROMA-LUC™ CBR68luc red light-emitting luciferase gene (Promega, Madison, Wis.) under the control of the CMV promoter is co-transfected with P2-green luciferase construct into HT116 colon cancer cells.

The transfected cells are distributed at approximately 50,000 cells per well into 384 well multiwell plates. Compounds from a compound library are added to the wells to a final concentration of 0.5 micomolar. A series of control wells for each cell type receive only buffer or compound solvent. Four hours after the addition of compound, the cell lysis/luciferase reagent buffer is added to each well and ten minutes later the signal from the luciferases is read at 544 nm (LEF1 P2 expression reporter) and 611 nm (control reporter).

Six hours after the addition of test compounds, the cells are assayed for luciferases by a luminometer that reads at the wavelengths of both luciferases, and the signal of the reporter gene greem-emitting luciferase is normalized to the value of the control gene red-emitting luciferase. Compounds having increased normalized luciferase activity with respect to control cells to the normalized luciferase activity to which no test compound was added are identified as compounds that upregulate the LEF1 P2 promoter.

### Example 4

### Alternative Promoter Use Assay

A reporter gene construct is made using the approximately 5.5 kb promoter region of the LEF1 gene includes both the P1 and P2 promoters as well as the first three exons of the gene. This region includes the P1 promoter, the first two exons of the LEF1 gene, the P2 promoter, and a portion of the third exon of the LEF1 gene.

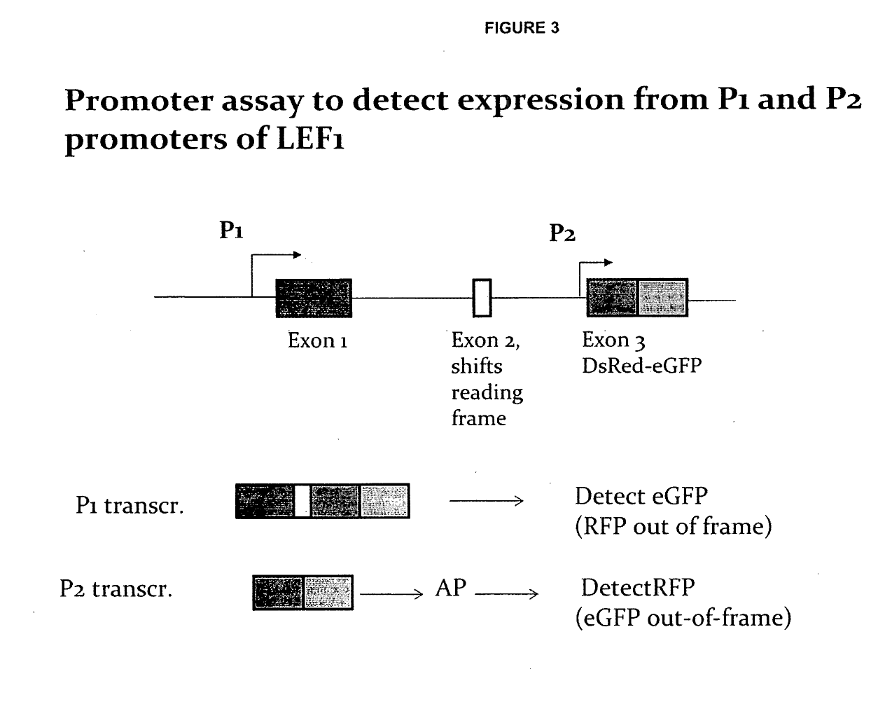

The construct includes in addition to the 5.5 kb dual promoter region, two fluorescent protein genes appended to exon 3 of the LEF1 gene: the dsRed gene and enhanced green fluorescent protein (eGFP) gene (see FIG. 3) which show promoter assay to detect expression from P₁ and P₂ promoters of LEF1. The dsREd and eGFP genes are juxtaposed such that they create a single open reading frame in reading frame 1 (the reading frame of the LEF1 gene) which translates an open reading frame of the dsRed gene that does not encode dsRed that is contiguous with the eGFP-encoding reading frame. Thus, translation of a gene transcribed from the P3 promoter that begins at exon 3 will translate a LEF1(exon3)-eGFP fusion protein, which is detectable by its green fluorescence.

Exon 2 of the construct has a single base deletion near its 3′end that changes the reading frame thereafter. When exon 2 is spliced to exon 3, DsRed is transcribed in its proper reading frame with the previous sequences, but GFP is out-of-frame, and includes stop codons in the DsRed frame, producing to a processed transcript that is translated to produce a LEF1-DsRed fusion protein. The first 100 bases of exon 3, immediately prior to the beginning of the DsRed gene, are mutated to remove any stop codons that would otherwise lead to a truncation of the translation product prior to the DsRed frame.

The dual promoter/dual reporter gene construct is made in the pLVX-Puro (Clontech, Mountain View, Calif.) “third generation” lentiviral vector, and lentivirus made by packaging cells is used to infect SW480 colon cancer cells. Stable integrants are selected for using puromycin.

Cell line NCM356-βcat, a cell line derived from normal (noncancerous) colon cells that have an integrated tet-inducible β-catenin gene, is infected with the same lentivirus and also selected for stable integration using puromycin.

For assays using the SW480 cells, the cells are seeded into 384 well plates (approximately 50,000 cells per well) and after 24 hours test compounds are added to the wells to a final concentration of 1 micromolar. A series of control well is maintained in which the cells do not receive test compound. After an additional 8 hours, the emission of RFP and GFP are read using a flourimeter to determine the relative amount of transcription from the P1 promoter with respect to the P2 promoter.

For assays using the NCM 356 normal colon cells, the cells are seeded into 384 well plates (approximately 50,000 cells per well) and after 16 hours, half of the cells are induced to express β-catenin with doxycycline. After an additional 8 hours, test compounds are added to the wells to a final concentration of 1 micromolar. A series of control wells is maintained for both β-catenin induced and non-induced cells, in which the cells do not receive test compound. After an additional 8 hours, the emission of RFP and GFP are read using a flourometer to determine the relative amount of transcription from the P2 promoter with respect to the P1 promoter.

After 12 hours, 24 hours, and 36 hours, the emissions from the wells are read again using the plate reader. Identification of wells in which the ratio of emissions from the red fluorescent protein and the green fluorescent protein have changed is used to identify test compounds that are candidates for drugs that modulate promoter use of LEF-1, and drugs that can modulate expression of the P2 promoter.

Some of the embodiments presented herein and further embodiments of the invention are illustrated in the appended pages.

1. A method for identifying a compound that modulates a cancer-associated alternative splicing process, comprising:
(a) providing a cell that comprises a nucleic acid construct, wherein the nucleic acid construct comprises a transcription unit comprising:
(i) a promoter
(ii) a first reporter gene and a second reporter gene, wherein the first reporter gene and the second reporter gene are differently detectable, and
(iii) an alternative splice module comprising at least three exons, wherein the alternative splice module is operably linked to the promoter, and wherein sequences of the exon-intron boundaries of the alternative splice module are derived from a gene that exhibits cancer-associated alternative splicing;
wherein a first alternative splicing event results in the splicing of the first exon to the second exon, resulting in the expression of the first reporter gene and the second reporter gene, and the second splicing event results in the splicing of the first exon to the third exon, resulting in the expression of the second reporter gene;
(b) contacting the cell with a test compound;
(c) detecting the signals from expression of the first reporter gene and the second reporter gene; and
(d) calculating a ratio of the expression of the first reporter gene to the second reporter gene and detecting a difference between the reporter gene expression ratio in the cell contacted with the test compound to the reporter gene expression ratio in a cell not contacted with the test compound, whereby a difference in the reporter gene expression ratio indicates that the test compound modulates a cancer-associated alternative splicing process.

2. The method of claim 1, wherein the first reporter gene is within the second exon and the second reporter gene is within the third exon, wherein a first alternative splicing event results in the splicing of the first exon to the second exon, resulting in the expression of the first reporter gene and the second reporter gene, and the second splicing event results in the splicing of the first exon to the third exon, resulting in the expression of the second reporter gene and not the first reporter gene.

3. The method of claim 2, wherein the first reporter gene and the second reporter gene are located in the third exon, wherein a first alternative splicing event results in the first reporter gene being in-frame and expressed and the second reporter gene being out-of-frame and not expressed and a second alternative splicing event results in the second reporter gene being in-frame and expressed and the first reporter gene being out-of-frame and not expressed.

4. The method of claim 3, wherein the first alternative splicing event results in the inclusion of exon 2 and the second alternative splicing event results in the exclusion of exon 2.

5. The method of any of claims 1-4, wherein the alternative splice module is derived from Ron.

6. The method of any of claims 1-4, wherein the alternative splice module is derived from Bcl-X.

7. The method of any of claims 1-4, wherein the alternative splice module is derived from a gene that affects Wnt signaling.

8. The method of claim 7, wherein the gene that affects Wnt signaling is a LEF/TCF gene.

9. The method of claim 8, wherein the gene that affects Wnt signaling is a LEF-1 gene.

10. The method of claim 9, wherein the alternative splice module comprises exon 10, exon 11, and exon 12 of the LEF1 gene.

11. The method of claim 10, wherein the protein affects Wnt signaling is a TCF-4 protein.

12. The method of claim 11, wherein the alternative splice module comprises exon 8, exon 9, and exon 10 of the TCF-4 gene.

13. The method of claim 1, wherein the alternative splice module comprises more than 3 exons.

14. The method of claim 13, wherein the alternative splice module comprises 4 exons.

15. The method of claim 13, wherein the alternative splice module comprises 5 exons.

16. The method of claim 13, wherein the alternative splice module comprises 6 or more exons.

17. A method for identifying a compound that modulates Wnt signaling, comprising:
(a) providing a cell that comprises a nucleic acid construct, wherein the nucleic acid construct comprises:
a promoter region of a gene that affects Wnt signaling, wherein the promoter regions comprise two alternative promoters, wherein the ratio of isoforms resulting from the two alternative promoters affects Wnt signaling;
two differently detectable reporter genes linked to the 3′ end of the alternative promoter region of the gene that affects Wnt signaling; wherein expression of the first reporter gene is the result of transcription from the first alternative promoter and expression of the second reporter gene is the result transcription from the second alternative promoter;
(b) contacting the cell with a test compound; and
(c) detecting a difference in the ratio of the signal from expression of the first reporter gene and the second reporter gene; and
(d) identifying a test compound that results in a difference in the ratio of transcription from the first and second promoters.

18. The method of claim 17, wherein the promoter region is the promoter region of the LEF1 gene, the TCF1 gene, or the Bcl-X gene.

19. The method of claim 18, wherein the promoter region is the promoter region of the LEF1 gene.

20. The method of any of the previous claims, wherein at least one of the first and second reporter genes is a luciferase gene, a beta galactosidase gene, a beta lactamase gene, a gene encoding CAT, a gene encoding a fluorescent protein, a gene encoding alkaline phosphatase, or a gene encoding thymidine kinase.

21. The method of claim 20, wherein at least one of the first and second reporter genes is a luciferase gene.

22. The method of claim 21, wherein at least one of the first and second reporter genes is a click beetle luciferase gene, a firefly luciferase gene, a Renilla luciferase gene, or a Gaussia luciferase gene.

23. The method of claim 20, wherein at least one of the first and second reporter genes is a fluorescent protein gene.

24. The method of claim 23, wherein the fluorescent protein gene is a green fluorescent protein gene, a yellow fluorescent protein gene a red fluorescent protein gene, an orange fluorescent protein gene, a cyan fluorescent protein gene, or a blue fluorescent protein gene.

25. The method of claim 20, wherein at least on of the first and second reporter genes is a gene encoding a secreted alkaline phosphatase, a secreted beta galactosidase, a secreted beta lactamase, or a secreted luciferase.

26. A method for identifying a compound that promotes transcription of the dominant negative form of LEF1, comprising:
(a) providing a cancerous cell that comprises a reporter gene regulated by the P2 promoter of the LEF1 gene;
(b) contacting the cell with a test compound; and
(c) detecting an increase in the signal from expression of the reporter gene in the cell contacted with the test compound as compared with the expression of the reporter gene in a control cell not contacted with the test compound to identify a compound that promotes transcription of the dominant negative form of LEF1.

27. The method claim 26, the reporter gene is a luciferase gene, a beta galactosidase gene, a beta lactamase gene, a gene encoding CAT, a gene encoding a fluorescent protein, a gene encoding alkaline phosphatase, or a gene encoding thymidine kinase.

28. The method of any of the previous claims, wherein the cells are cancer cells.

29. The method of claim 28, wherein the cancer cells are colon cancer cells, leukemia cells, lymphoma cells, melanoma cells, breast cancer cells, prostate cancer cells, hepatocarcinoma cells, or head and neck cancer cells.

30. The method of claim 29, wherein the cells are colon cancer cells, leukemia cells, or lymphoma cells.

31. The method of claim 30, wherein the cells are leukemia cells.

32. The method of claim 31, wherein the cells are Jurkat cells or K562 cells.

33. The method of claim 28, wherein the cells are colon cancer cells.

34. The method of claim 28, wherein the cells are SW48, SW480, SW116, CaCO2, DLD1, Colo320, Colo205, LS174T, HT-29, or HT-116 cells.

35. The method of any of the previous claims, wherein the cells are noncancerous cells.

36. The method of claim 35, wherein the cells are HEK 293 cells, HeLa cells, COS cells, CHO cells, 3T3 cells.

37. The method of claim 35, wherein the cells are noncancerous intestinal epithelial cells, noncanerous colon cells, noncancerous lymphocytes, noncancerous epithelial cells, noncancerous breast cells, noncancerous prostate cells, or noncancerous hepatocytes.

38. The method of claim 37, wherein the cells are noncancerous intestinal epithelial cells.

39. The method of claim 38, wherein the cells are normal human large intestinal epithelial cells (NHLIEC).

40. A method for identifying a cancer-specific isoform sequence of a protein, comprising:
(a) comparing RNA transcripts of wnt-related genes or cDNA generated from RNA transcripts of wnt-related genes isolated from cancer cells and normal cells of the same cell type; and
(b) identifying one or more exons uniquely present in RNA transcripts or cDNA generated from RNA transcripts of the cancer cells to identify at least one cancer-specific isoform sequence of a wnt-related protein.

41. The method of claim 40, wherein the RNA transcripts are compared by comparing databases of expressed genes or expressed sequence tags (ESTs).

42. A method for identifying a cancer-specific epitope of a wnt-related protein, comprising:
(a) performing tandem mass spectrometry on proteins isolated from cancer cells and on proteins isolated from normal cells of the same cell type;
(b) identifying one or more protein sequences of one or more wnt-related proteins uniquely present in the cancer cells to identify a cancer-specific sequence of a wnt-related protein.

43. A method of obtaining an antibody specific to an isoform of a wnt-related protein that is present in cancer cells but not present in normal cells, comprising:
identifying an amino acid sequence of a wnt-related protein isoform that is uniquely present in cancer cells;
expressing the amino acid sequence; and
generating an antibody to the amino acid sequence to obtain an antibody that binds to an isoform of a wnt-related protein that is present in cancer cells but not present in normal cells.

44. The method of claim 43, wherein the antibody is a monoclonal antibody.

45. The method of claim 43, wherein the antibody is a polyclonal antibody.

46. The method of claim 43, wherein the antibody is a humanized antibody.

47. An antibody specific to an isoform of a wnt-related protein that is present in cancer cells but not present in normal cells, wherein the antibody does not specifically bind to a protein in normal cells.
